(19) United States
(12) Patent Application Publication
(10) Pub. No.: US 2002/0193828 A1
(43) Pub. Date: Dec. 19, 2002

(54) Endovascular filter

(75) Inventors: Dennis Griffin, Englewood, CO (US); Arne Molgaard-Nielsen, Copenhagen (DK); Anthony O. Ragheb, West LaFayette, IN (US); Raymond B. Leonard, Bloomington, IN (US)

Correspondence Address: BRINKS HOFER GILSON & LIONE, P.O. Box 10395, Chicago, IL, 60610, US

(73) Assignee: COOK INCORPORATED

(21) Appl. No.: 10/172,725

(22) Filed: Jun. 14, 2002

Related U.S. Application Data
(60) Provisional application No. 60/298,803, filed on Jun. 14, 2001.

(51) Int. Cl. A61M 29/00

(52) U.S. Cl. 606/200

(57) ABSTRACT

Endovascular filter (10) including a plurality of struts (14) with distal ends (18) adapted to anchor the filter to the vessel wall after deployment, such as by having barbs (20), the filter being adapted to be retrieved if desired. Strut distal ends (18) are coated with an antiproliferative agent (40) that inhibits the ingrowth of tissue around the filter, thereby permitting the filter to be retrieved and removed atraumatically after a prolonged period of time, thus extending the useful life of the retrievable filter. Optionally, the proximal end (22) of the filter may also be so coated, or the entire filter.

### TECHNICAL FIELD

The present invention relates to medical devices and more particularly to endovascular filters.

### BACKGROUND OF THE INVENTION

In a trauma patient, orthopedic surgery patient, or neuro patient, where the patient is bedridden and not moving, clot frequently forms in the leg veins. Such clot becomes a serious risk of pulmonary embolism if it breaks loose. Recognition of this occurrence has led to the development of vena cava filters which provide protection from migrating clot. While many such filters are permanently deployed in the patient, temporary filters are known that are to be removed when it is determined that the patient is free of the risk of pulmonary embolism. Additionally, retrievable filters are known which may optionally be removed from the patient, if it is determined that the patient is free of the risk of pulmonary embolism within a short period of time after deployment. After deployment of a filter in the patient, proliferating intimal cells begin to grow around the filter struts; after a length of time, such ingrowth prevents removal of the filter without risk of trauma whereafter the filter must remain in the patient. Normally, removal of a filter is only advisable within a couple of weeks after implantation due to intimal proliferation that irreversibly anchors the filter to the vessel wall. See, for example, SCVIR March 2001, San Antonio, Tex., USA, Scientific Session 25 Abstract No. 194, Gimeno, M. S., et al.

In U.S. Pat. No. 5,133,733, a collapsible filter is disclosed that is implantable in a blood vessel of a patient, and in particular in the inferior vena cava. Such filters are utilized during endovascular procedures to entrap thrombi or emboli in the blood that flows through a vein and prevent them from reaching the lungs of a patient and thereby cause pulmonary embolization. Such filters are particularly, but not exclusively, concerned with the inferior vena cava, and have legs or similar structures that anchor to the vessel wall at the desired placement site. Other filters are disclosed in U.S. Pat. Nos. 3,540,431; 3,952,747; 4,425,908 and 4,619,246.

In the first-mentioned patent, a collapsible filter is provided that has limited axial length for facilitating the insertion procedure, with a moderate reduction of the blood flow area of the vein, and in its collapsed state the filter is concentrated into a slender and very narrow bundle of filter elements allowing for a correspondingly slender and narrow insertion catheter. In the expanded condition, four legs extend from an apical hub whereat they are joined together by a ferrule, and each leg of the filter comprises a central element, bent into a smooth quasi-halfsinusoidal form, and two substantially symmetrical curved side elements extending on either side of the central element are joined to the hub and to an eyelet surrounding the central element along its length that is slidable along the central element.

The filter of U.S. Pat. No. 5,133,733 as a whole may be folded to a collapsed condition having an outer diameter only about as large as the thicknesses of the metal central and side elements, and then is unfolded from a collapsed insertion condition in which the central elements and side elements of all legs forms a narrow bundle for arrangement in a catheter-like insertion instrument, into a tulip-like filter configuration with the side elements interposed between the central elements of the legs to assume the shape of an apertured solid of evolution with one pointed end at the apical hub. At the free end of each leg central element is a reversely turned anchoring hook engageable with the vessel wall for anchoring the filter in place. In the unfolded tulip-like configuration, the distal ends of the filter legs, both the central and side elements, will engage the wall of the vein along a certain length, minimizing the risk of perforation of the wall, and is said to provide an optimum possibility for filter ingrowth in the vein wall and thereby an optimum long term security against migration of the filter. If the filter needs to be removed after more than fourteen days, the filter ingrowth is an undesirable effect.

It is therefore desired to provide a vena cava filter that is adapted to be removable from its deployed location in a vessel of a patient without trauma to the tissue of the vessel wall and without risk of tearing of intimal tissue which could cause embolization.

It is further desired to provide such a retrievable filter that is adapted for extended retrieval time in a patient, again without risk of trauma.

### SUMMARY OF THE INVENTION

The foregoing problem is solved and a technical advance is achieved in an illustrative endovascular filter for retrievable deployment in a blood vessel of a patient. A plurality of struts extend and diverge from an apical hub at a proximal end to respective distal ends adapted to anchor to the vessel wall when expanded and deployment at a treatment site in a blood vessel of a patient, and lengths of the distal ends of the struts are engageable with and against the vessel wall when deployed. The distal end lengths, and preferably the anchoring sections also, are coated with an antiproliferative agent or bioactive material that prevents or minimizes tissue growth. One such particularly useful bioactive material is paclitaxel, a drug known to have cytostatic properties and that has been shown to inhibit vascular smooth cell migration and proliferation contributing to neointimal hypoplasia.

In an additional aspect, it is preferable to also coat the proximal end of the filter with the antiproliferative agent. Ingrowth would be inhibited were the proximal end to enter into engagement with the vessel wall when the filter becomes misaligned. Likewise, other surface portions of the hub body and side members between the distal and proximal filter ends are preferably coated, were these portions to engage the vessel wall upon misalignment, since the vessel wall may locally protrude inwardly from a linear configuration relative to the filter.

### BRIEF DESCRIPTION OF THE DRAWINGS

An embodiment of the present invention will now be described by way of example with reference to the accompanying drawings, in which:

FIG. 1 discloses an elevation view of an endovascular filter of the present invention in a fully expanded condition;

FIG. 2 is an end view of the expanded filter;

FIG. 3 is an enlargement of one wall-engaging strut distal end that has been treated with an antiproliferative agent;

FIG. 4 is a cross-sectional view through a coated strut end;

FIG. 5 is a view of the filter of FIG. 1 upon deployment in the vena cava; and

FIG. 6 illustrates the filter of FIG. 1 being deployed from its delivery system, in the arrangement suitable for a jugular vein approach to the treatment site.

### DETAILED DESCRIPTION

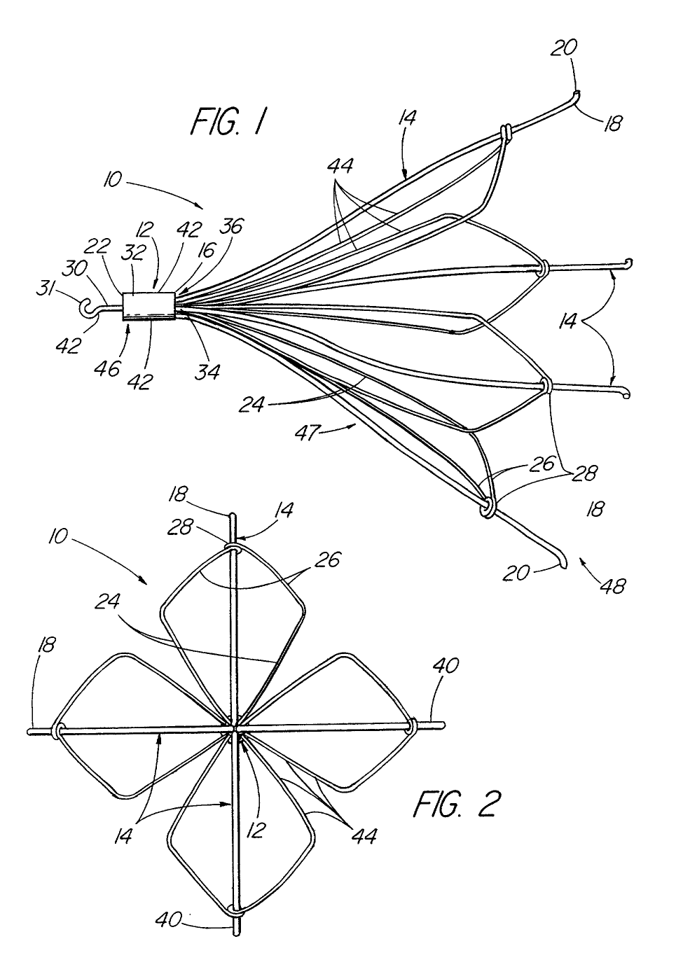

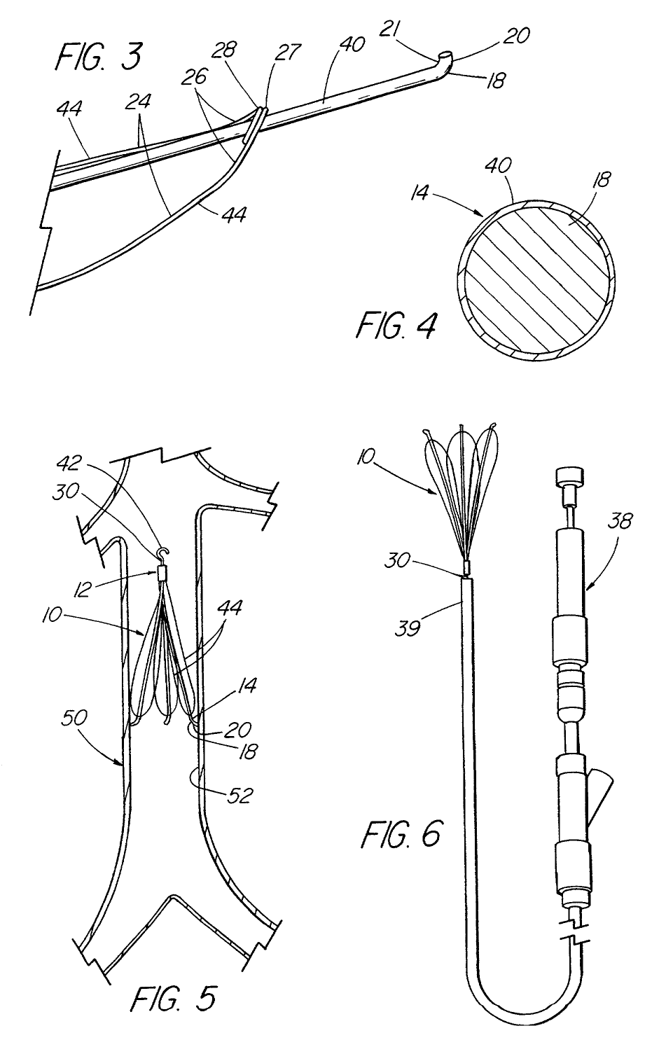

Vena cava filter 10 is shown in FIGS. 1 to 3 in its fully expanded condition to have a proximal portion 46, a medial portion 47 and a distal portion 48. An apical hub body 12, in the proximal portion 46 of the filter 10, has a first or distal end 16 and a second or proximal end 22. A plurality of struts 14 have proximal ends 34 that are secured to the distal end 16 of hub body 12 and have distal end portions 18 that have anchoring sections 20. The struts 14 divergingly extend distally from the distal end 16 of hub body 12. The second or proximal end 22 of hub body 12 has a retrieval section 30 extending therefrom that terminates in a hook 31. The specific embodiment of the filter 10 that is illustrated is shown to have pairs of side elements 24 having proximal ends 36 that are connected to the first end 16 of the hub body 12, each pair of which is associated with a strut 14. The side elements 24 also extend distally in diverging pairs from first end 16 of the hub body 12 and includes distal end portions 26 that converge at 28 and are slidably connected to their associated strut 14. (see FIG. 3) The connection of side elements 24 to the struts 14 preferably being an eyelet 27 that surrounds the strut 14 and is slidable along the strut 14.

Anchoring sections 20 preferably are formed as short hooks 21 that are adapted to press slightly into the wall 52 of a vessel 50 (see FIG. 5) at the deployment site to prevent movement in the direction of blood flow. Apical hub body 12 is adapted to be engaged and retrieved by a retrieval device such as a snare, which can be remotely manipulated to snatch the hook 31 of the retrieval section 30. The retrieval section 30 extends from the second or proximal end 22 of the hub body 12. A ferrule 32 secures the proximal ends 34 of struts 14 and proximal ends 36 of side elements 24, to the hub body 12.

FIG. 6 illustrates the filter 10 being deployed from the catheter 39 of delivery and deployment system 38; the filter has an outermost dimension when in a collapsed state essentially no greater than the combined thicknesses of the hub body, proximal ends 34, 36 of struts 14 and side elements 24, and ferrule 32 therearound, to facilitate assembly into the delivery and deployment system 38 and deployment therefrom. The filter 10 must also be capable of collapsing back to this size so that it can be “swallowed” by a sheath of a retrieval device after the retrieval device snares the hook 31 of the retrieval section 30 during removal from the patient. FIG. 6 shows the arrangement suitable for a jugular vein approach to the treatment site. For a femoral approach, the filter would be reversed in orientation, with the retrieval section 30 being the forwardmost section during delivery. A quite similar filter structure is disclosed in U.S. Pat. No. 5,133,733 and a similar product is sold by William Cook Europe ApS, Bjaeverskov, Denmark as the GÜNTHER TULIP™ Filter, which is designed to be retrievable. Delivery of a filter such as that disclosed in U.S. Pat. No. 5,133,733 is described in detail in U.S. Pat. No. 5,324,304.

At some point after implantation, many patients may resume their mobility and no longer need protection from migrating clot. The current maximum retrieval time after implantation for the GÜNTHER TULIP filter is fourteen days; thereafter, the filter grows into the caval wall, or more precisely, strands of organized thrombus grow around the struts and anchoring sections.

In accordance with the present invention, the distal end sections 18 of struts 14 as well as their anchoring sections 20, are coated with an antiproliferative or antiinflammatory agent 40, shown in FIG. 4. Coating 40 inhibits or prevents the ingrowth of tissue to and around the distal end portions 18 and anchoring sections 20, at least for an extended length of time after placement, such as for four weeks or more, thereby substantially extending the maximum retrieval time for the filter. This inhibition of ingrowth extends the protection period for the immobile patient, and yet still preserves the eventual retrievability of the filter.

Occasionally an emplaced filter will become misaligned within the vessel, to the extent that the second or proximal end 22 of the hub body 12 will become engaged with the vessel wall 52. While retrieval is still possible although it is more complicated to establish engagement by the retrieval device with the hook 31 of retrieval section 30, it is also desirable to provide a coating of the antiproliferative or antiinflammatory agent 40 to those portions of the filter that may enter into contact with the vessel wall such as portions 42 of the second or proximal end 22 of the hub body 12 including the retrieval section 30 (FIG. 1). Similarly, it may be desirable to provide a coating of agent 40 onto surface portions in the medial portion 44 of the filter including portions of the side elements 24 and struts 14 that are spaced from the distal 48 and proximal 46 filter ends, since the vessel wall 52 may locally “protrude” inwardly because it may not remain truly coaxial around the filter.

One such agent is dexamethasone and related compounds. Another is paclitaxel. Coating of an implantable medical device such as a stent, with a bioactive material, such as paclitaxel, is disclosed in U.S. Pat. No. 6,299,604. It has become well-established that paclitaxel in particular has cytotoxic properties when provided in proper dosages and concentrations, as described in U.S. Pat. No. 6,299,604, and in lower dosages and concentrations would be considered at least cytostatic and therefore able to inhibit neointimal growth, and hence very useful in preventing or inhibiting restenosis.

The coating may be applied by numerous methods, including but not limited to, spraying, dipping, soaking, painting with a brush or similar tool. In the present embodiment the method of coating was spraying as a fine mist. For simplification of fabrication, the entire filter may be so coated.

An excipient (e.g., matrix, binder, carrier, polymer, membrane) may be associated with the active agent and may be under the bioactive layer, over the bioactive layer, mixed with the bioactive layer, or any combination thereof. The excipient material may include, but is not limited to parylene, a cellulose based polymer or a naturally occurring basement membrane material such as Small Intestine Submucosa (SIS).

In the present embodiment, because paclitaxel has low water solubility, no excipient need be used, and the coating may be entirely paclitaxel. The coated device should be handled as gently as possible with minimum scraping, abrading, rubbing, soaking or other physical challenge.

A wide range of other bioactive materials can be delivered by the filter, as set forth in U.S. Pat. No. 6,096,070. Accordingly, it is preferred that the bioactive material includes at least one of heparin, covalent heparin, or another thrombin inhibitor, hirudin, hirulog, argatroban, D-phenylalanyl-L-polyL-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; Hytrin® or other antihypertensive agents; an antimicrobial agent or antibiotic; aspirin, ticlopidine, a glycoprotein Ilbilila inhibitor or another inhibitor of surface glycoprotein receptors, or another antiplatelet agent; colchicine or another antimitotic, or another microtubule inhibitor, dimethyl sulfoxide (DMSO), a retinoid or another antisecretory agent; cytochalasin or another actin inhibitor; or a remodelling inhibitor; deoxyribonucleic acid, an antisense nucleotide or another agent for molecular genetic intervention; methotrexate or another antimetabolite or antiproliferative agent; tamoxifen citrate, Taxol® or the derivatives thereof, or other anti-cancer chemotherapeutic agents; dexamethasone, dexamethasone sodium phosphate, dexamethasone acetate or another dexamethasone derivative, or another anti-inflammatory steroid or non-steroidal antiinflammatory agent; cyclosporin or another immunosuppressive agent; trapidal (a PDGF antagonist), angiopeptin (a growth hormone antagonist), angiogenin, a growth factor or an anti-growth factor antibody, or another growth factor antagonist; dopamine, bromocriptine mesylate, pergolide mesylate or another dopamine agonist; ⁶⁰Co (5.3 year half life), ¹⁹²Ir (73.8 days), ³²P (14.3 days), ¹¹¹In (68 hours), ⁹⁰Y (64 hours), ^{99m}TC (6 hours) or another radiotherapeutic agent; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21 -aminosteroid (lasaroid) or another free radical scavenger, iron chelator or antioxidant; a ¹⁴C-, ³H-, ¹³¹I-, ³²P- or ³⁶S-radiolabelled form or other radiolabelled form of any of the foregoing; estrogen or another sex hormone; AZT or other anti polymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents; gene therapy agents; and enalapril and other prodrugs; Proscar®, Hytrin® or other agents for treating benign prostatic hyperplasia (BHP) or a mixture of any of these; and various forms of small intestine submucosa (SIS).

In a particularly preferred aspect, the layer of bioactive material contains from about 0.1 to 10.0 μg/mm², more preferably about 1.0 to 5.0 μg/mm², and in the present embodiment was about 3.0 μg/mm² of the gross surface area of the structure. “Gross surface area” refers to the area calculated from the gross or overall extent of the structure, and not necessarily to the actual surface area of the particular shape or individual parts of the structure. In other terms, about 100 μg to about 300 μg of drug per 0.001 inch of coating thickness may be contained on the device surface.

1. A collapsible vena cava filter for introduction into a blood vessel of a patient comprising:
an apical hub;
a plurality of struts secured to and diverging from said apical hub, each of said plurality of struts terminating in holding mechanisms that engage the walls of the blood vessel to secure the filter in a selected location therein;
filter media connected to said struts and spanning the space between the struts;
a bioactive coating applied to the surfaces of said filter to prevent the growth of tissue that would interfere with removal of the filter as well as medicate the patient; and
wherein said layer of bioactive material contains from about 0.1 to 10.0 μg/mm², more preferably about 1.0 to 5.0 μg/mm², and most preferred about 3.0 μg/mm² of the coated surface area.

2. A collapsible vena cava filter for introduction into a blood vessel of a patient as set forth in claim 1 wherein the bioactive coating is paclitaxel.

3. A collapsible vena cava filter for introduction into a blood vessel of a patient as set forth in claim 1 wherein the bioactive coating is dexamethasone or related compounds.

4. A collapsible vena cava filter for introduction into a blood vessel of a patient as set forth in claim 1 wherein the bioactive coating is applied to surfaces of the apical hub, struts and filter media that could engage the vessel wall.

5. A collapsible vena cava filter for introduction into a blood vessel of a patient as set forth in claim 1 wherein the bioactive coating is applied to the gross surfaces area of the filter.

6. A collapsible vena cava filter for introduction into a blood vessel of a patient as set forth in claim 1 wherein an excipient may be associated with said bioactive coating.

7. A collapsible vena cava filter for introduction into a blood vessel of a patient as set forth in claim 4 or 5 wherein the bioactive coating is paclitaxel.

8. A collapsible vena cava filter for introduction into a blood vessel of a patient as set forth in claim 1 or 4 or 5 wherein the bioactive coating includes at least one of heparin, covalent heparin, or another thrombin inhibitor, hirudin, hirulog, argatroban, D-phenylalanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; Hytrin® or other antihypertensive agents; an antimicrobial agent or antibiotic; aspirin, ticlopidine, a glycoprotein IIb/IIIa inhibitor or another inhibitor of surface glycoprotein receptors, or another antiplatelet agent; colchicine or another antimitotic, or another microtubule inhibitor, dimethyl sulfoxide (DMSO), a retinoid or another antisecretory agent; cytochalasin or another actin inhibitor; or a remodelling inhibitor; deoxyribonucleic acid, an antisense nucleotide or another agent for molecular genetic intervention; methotrexate or another antimetabolite or antiproliferative agent; tamoxifen citrate, Taxol® or the derivatives thereof, or other anti-cancer chemotherapeutic agents; dexamethasone, dexamethasone sodium phosphate, dexamethasone acetate or another dexamethasone derivative, or another anti-inflammatory steroid or non-steroidal antiinflammatory agent; cyclosporin or another immunosuppressive agent; trapidal (a PDGF antagonist), angiopeptin (a growth hormone antagonist), angiogenin, a growth factor or an anti-growth factor antibody, or another growth factor antagonist; dopamine, bromocriptine mesylate, pergolide mesylate or another dopamine agonist; ⁶⁰Co (5.3 year half life), ¹⁹²Ir (73.8 days), ³²P (14.3 days), ¹¹¹In (68 hours), ⁹⁰Y (64 hours), ^{99m}Tc (6 hours) or another radiotherapeutic agent; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21 -aminosteroid (lasaroid) or another free radical scavenger, iron chelator or antioxidant; a ¹⁴C-, ³H-, ¹³¹I-, ³²P- or ³⁶S-radiolabelled form or other radiolabelled form of any of the foregoing; estrogen or another sex hormone; AZT or other anti polymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents; gene therapy agents; and enalapril and other prodrugs; Proscar®, Hytrin® or other agents for treating benign prostatic hyperplasia (BHP) or a mixture of any of these; and various forms of small intestine submucosa (SIS).

9. A collapsible vena cava filter for introduction into a blood vessel of a patient comprising:
an apical hub;
a plurality of struts secured to and diverging from said apical hub, each of said plurality of struts terminating in holding mechanisms that engage the walls of the blood vessel to secure the filter in a selected location therein;
filter media connected to said struts and spanning the space between the struts;
a bioactive coating applied to the surfaces of said filter to prevent the growth of tissue that would interfere with removal of the filter as well as medicate the patient; and
wherein said layer of bioactive material contains about 100 μg to about 300 μg of drug per 0.001 inch of coating thickness.

10. A collapsible vena cava filter for introduction into a blood vessel of a patient as set forth in claim 9 wherein the bioactive coating is paclitaxel.

11. A collapsible vena cava filter for introduction into a blood vessel of a patient as set forth in claim 9 wherein the bioactive coating is dexamethasone or related compounds.

12. A collapsible vena cava filter for introduction into a blood vessel of a patient as set forth in claim 9 wherein the bioactive coating is applied to surfaces of the apical hub, struts and filter media that could engage the vessel wall.

13. A collapsible vena cava filter for introduction into a blood vessel of a patient as set forth in claim 9 wherein the bioactive coating is applied to the gross surfaces area of the filter.

14. A collapsible vena cava filter for introduction into a blood vessel of a patient as set forth in claim 12 or 13 wherein the bioactive coating is paclitaxel.

15. A collapsible vena cava filter for introduction into a blood vessel of a patient as set forth in claim 9 or 12 or 13 wherein the bioactive coating includes at least one of heparin, covalent heparin, or another thrombin inhibitor, hirudin, hirulog, argatroban, D-phenylatanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; Hytrin® or other antihypertensive agents; an antimicrobial agent or antibiotic; aspirin, ticlopidine, a glycoprotein IIb/IIIa inhibitor or another inhibitor of surface glycoprotein receptors, or another antiplatelet agent; colchicine or another antimitotic, or another microtubule inhibitor, dimethyl sulfoxide (DMSO), a retinoid or another antisecretory agent; cytochalasin or another actin inhibitor; or a remodelling inhibitor; deoxyribonucleic acid, an antisense nucleotide or another agent for molecular genetic intervention; methotrexate or another antimetabolite or antiproliferative agent; tamoxifen citrate, Taxol® or the derivatives thereof, or other anti-cancer chemotherapeutic agents; dexamethasone, dexamethasone sodium phosphate, dexamethasone acetate or another dexamethasone derivative, or another anti-inflammatory steroid or non-steroidal antiinflammatory agent; cyclosporin or another immunosuppressive agent; trapidal (a PDGF antagonist), angiopeptin (a growth hormone antagonist), angiogenin, a growth factor or an anti-growth factor antibody, or another growth factor antagonist; dopamine, bromocriptine mesylate, pergolide mesylate or another dopamine agonist; ⁶⁰Co (5.3 year half life), ¹⁹²Ir (73.8 days), ³²P (14.3 days), ¹¹¹In (68 hours), ⁹⁰Y (64 hours), ^{99m}Tc (6 hours) or another radiotherapeutic agent; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21-aminosteroid (lasaroid) or another free radical scavenger, iron chelator or antioxidant; a ¹⁴C-, ³H-, ¹³¹I-, ³²P- or ³⁶S-radiolabelled form or other radiolabelled form of any of the foregoing; estrogen or another sex hormone; AZT or other anti polymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents; gene therapy agents; and enalapril and other prodrugs; Proscar®, Hytrin® or other agents for treating benign prostatic hyperplasia (BHP) or a mixture of any of these; and various forms of small intestine submucosa (SIS).

16. A collapsible filter for introduction into a blood vessel of a patient, said collapsible filter having a proximal portion, a medial portion and a distal portion, comprising:
an apical hub, in the proximal portion of said filter, having a first or distal end and a second or proximal end;
a plurality of struts having proximal end and distal end portions, the proximal ends of said plurality of struts being secured to the first or distal end of said apical hub and diverging distally and outwardly therefrom, and each of said struts having an outwardly turned hook at their distal ends;
a pair of side element associated with each of said struts, each side element having a proximal portion and a distal portion, the proximal end of the proximal portions being secured to the first or distal end of said apical hub and diverging distally and outwardly therefrom such that the associated strut lies between the pair of side elements, the distal portion of each side element diverging inwardly toward said associated strut such that the distal ends of the pair of side elements meet and form an eyelet through which the associated strut passes in a sliding relationship, whereby the filter as a whole may be unfolded from a collapsed insertion condition in which the struts and side elements form a narrow bundle for arrangement in a catheter like insertion instrument into an open tulip like filter configuration with the side elements interoposed between the struts;
a deployment and retrieval section secured to and extending proximately from the second or proximal end of said apical hub;
a bioactive coating applied to the surfaces of said filter to prevent the growth of tissue that would interfere with removal of the filter as well as medicate the patient; and
wherein the bioactive coating contains from about 0.1 to 10.0 μg/mm², more preferably about 1.0 to 5.0 μg/mm², and most preferred about 3.0 μg/mm² of the coated surface area.

17. A collapsible filter for introduction into a blood vessel as set forth in claim 16, wherein:
said bioactive coating is applied to the distal end portion of the struts and their hooks to prevent the ingrowth of tissue to and therearound.

18. A collapsible filter for introduction into a blood vessel as set forth in claim 17, wherein:
said bioactive coating is also applied to said first or distal end of the apical hub and the deployment and retrieval section that is secured to the apical hub to prevent the ingrowth of tissue to and therearound.

19. A collapsible filter for introduction into a blood vessel as set forth in claim 16, wherein:
said bioactive coating is applied to the gross surface area of the filter to prevent the ingrowth of tissue to and therearound.

20. A collapsible filter for introduction into a blood vessel as set forth in each of claim 16 or 17 or 19 wherein:
said bioactive coating is dexamethasone.

21. A collapsible filter for introduction into a blood vessel as set forth in each of claims 16 or 17 or 19 wherein:
said bioactive coating is pacilitaxel.

22. A collapsible filter for introduction into a blood vessel of a patient as set forth in any of claims 15 or 16 or 18 wherein the layer of bioactive coating contains about 100 μg to about 300 μg of drug per 0.001 inch of coating thickness.

23. A collapsible filter for introduction into a blood vessel of a patient as set forth in any of claims 16 or 17 or 19 wherein the layer of bioactive coating includes at least one of heparin, covalent heparin, or another thrombin inhibitor, hirudin, hirulog, argatroban, D-phenylalanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; Hytrin® or other antihypertensive agents; an antimicrobial agent or antibiotic; aspirin, ticlopidine, a glycoprotein IIb/IIIa inhibitor or another inhibitor of surface glycoprotein receptors, or another antiplatelet agent; colchicine or another antimitotic, or another microtubule inhibitor, dimethyl sulfoxide (DMSO), a retinoid or another antisecretory agent; cytochalasin or another actin inhibitor; or a remodelling inhibitor; deoxyribonucleic acid, an antisense nucleotide or another agent for molecular genetic intervention; methotrexate or another antimetabolite or antiproliferative agent; tamoxifen citrate, Taxol® or the derivatives thereof, or other anti-cancer chemotherapeutic agents; dexamethasone, dexamethasone sodium phosphate, dexamethasone acetate or another dexamethasone derivative, or another anti-inflammatory steroid or non-steroidal antiinflammatory agent; cyclosporin or another immunosuppressive agent; trapidal (a PDGF antagonist), angiopeptin (a growth hormone antagonist), angiogenin, a growth factor or an anti-growth factor antibody, or another growth factor antagonist; dopamine, bromocriptine mesylate, pergolide mesylate or another dopamine agonist; ⁶⁰Co (5.3 year half life), ¹⁹²Ir (73.8 days), ³²P (14.3 days), ¹¹¹In (68 hours), ⁹⁰Y (64 hours), ^{99m}Tc (6 hours) or another radiotherapeutic agent; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21-aminosteroid (lasaroid) or another free radical scavenger, iron chelator or antioxidant; a ¹⁴C-, ³H-, ¹³¹I-, ³²P- or ³⁶S-radiolabelled form or other radiolabelled form of any of the foregoing; estrogen or another sex hormone; AZT or other anti polymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents; gene therapy agents; and enalapril and other prodrugs; Proscar®, Hytrin® or other agents for treating benign prostatic hyperplasia (BHP) or a mixture of any of these; and various forms of small intestine submucosa (SIS).
